(19) United States
(12) Patent Application Publication
(10) Pub. No.: US 2004/0077941 A1
(43) Pub. Date: Apr. 22, 2004

(54) Method and system for image improvement with ECG gating and dose reduction in CT imaging

(75) Inventors: Shankara Bonthu Reddy, Cedarburg, WI (US); Priya Gopinath, North Arlington, NJ (US); Kishore C. Acharya, Brookfield, WI (US); Jianying Li, New Berlin, WI (US)

Correspondence Address: CANTOR COLBURN, LLP, 55 GRIFFIN ROAD SOUTH, BLOOMFIELD, CT, 06002

(73) Assignee: GE Medical Systems Global Technology Company, LLC, Waukesha, WI (US)

(21) Appl. No.: 10/065,462

(22) Filed: Oct. 21, 2002

(51) Int. Cl. A61B 5/04; A61B 5/05

(52) U.S. Cl. 600/428; 600/509

(57) ABSTRACT

A method and system for associating ECG waveform data with medical imaging data using ECG gating for dose reduction and image improvement by generating the ECG waveform data using an electrocardiogram device. The ECG data is first validated and then QRS complexes are detected using a detection function. An underlying cardiac rhythm based on the detected QRS complexes is analyzed and an even number N of substantially normally shaped consecutive QRS complexes are selected. An RR interval between consecutive QRS complexes is computed to yield N−1 intervals. Duration of a representative cardiac cycle by averaging at least a plurality of the N−1 intervals is determined. Once a representative cardiac cycle is determined, a method to control power and improve image quality with the presence of patient's having arrhythmias is disclosed.

### BACKGROUND OF THE INVENTION

The invention relates to cardiac imaging in computed tomography (CT), magnetic resonance (MR) imaging, nuclear imaging, ultrasound, and other imaging modalities, and more particularly, to an apparatus and a method for use in cardiac gating to improve image quality and for dose reduction in prospective ECG gating used in imaging modalities where dose to patient is a concern. Although the method is applicable to cardiac imaging by all modalities, the following description is given for cardiac imaging by a CT system.

Cardiac imaging includes coronary artery imaging, imaging for determining the cardiac function and perfusion and identification of walls of the heart chambers and valvular structures. Common to all of the above applications of cardiac imaging and common to all imaging modalities (CT, MR, Ultrasound, Nuclear, etc.) is the need for proper gating of the images to certain phases of the cardiac cycle. When the heart rate is low, the intervals between the heart beats are nearly constant and each mechanical contraction of the heart is nearly the same (e.g., sinus rhythm with heart rate less than 65 beats), cardiac images taken by any modality will be of high diagnostic quality. However when the heart rate changes suddenly and intermittently due to arrhythmias, quality of the image degrades appreciably. Solutions to address these problems and thus improve the image quality are described here with reference to cardiac CT imaging. The invention described here, although described with reference to cardiac CT imaging, is equally applicable to other imaging modalities such as MR, Ultrasound, PET, Nuclear and thermal imaging.

In current CT systems, in order to appropriately produce CT images of the heart's chambers (myocardium), inter-chamber valves and coronary vasculature, it is necessary to acquire CT radiograph data while the heart is at a certain position that is substantially spatially stationary. This requires that the heart rate of the patient be extremely slow, which is not clinically viable, or that the speed of the gantry be extremely high, which is not technically viable. In the past, a few different techniques have been employed in attempts to solve this problem. One technique, known as prospective gating, uses the ECG (electrocardiogram) signal of the heart to trigger data acquisition by the detector array at points in time when the heart is fairly stationary (typically during diastole) so that the radiographs used to reconstruct the image correspond to instants in time when the heart is fairly stationary. Another technique, known as retrospective gating, measures the ECG signal while acquiring CT radiograph data and then retrospectively selects the data that corresponds to a point in time of the ECG signal when the heart is fairly stationary.

With both of these techniques, only CT radiograph data that corresponds to a certain time interval during which the heart is substantially spatially stationary is used in reconstructing the CT images. Therefore, during reconstruction, both techniques only use CT radiograph data corresponding to certain view angles, i.e., neither technique uses measured CT radiograph data at all view angles of the CT gantry. Also, both techniques use CT radiograph data obtained during a particular window in time as the heart is moving. Consequently, the CT reconstructions may suffer from motion artifacts and/or limited view angle artifacts.

One disadvantage of retrospective ECG gating for cardiac imaging in helical CT scanners includes patient exposure to a higher dose of X-ray radiation. To reduce the dose in cardiac imaging, prospective gating is often used where the X-ray tube is powered only during a certain period of the cardiac cycle and images are acquired (image acquisition period). In order to select a suitable window for image acquisition during the most quiescent period (i.e., least mechanical motion of heart), duration of the average cardiac cycle is estimated from a small number of RR intervals (time intervals between R waves or onsets of the adjacent QRS complexes) from the patient's ECG before the actual scan begins. During the scan, the X-ray tube is typically powered for an imaging window (measured in milliseconds) of (⅔*gantry speed*1000)+(M−1)*T where:

M=Number of images/cardiac cycle

Gantry speed is about 0.5, 0.8, or 1 sec/revolution

T=Time Interval (either 50 or 100 ms).

The imaging window is centered typically between about 60% and about 80% of duration of a representative cardiac cycle (‘phase’). Different window widths and phases, including multiple phases, can be selected based on the choice of scanning protocol.

Presence of arrhythmias in a patient's ECG, particularly premature heart beats (such as premature atrial beats (PABs), premature ventricular beats (PVBs), atrial fibrillation, and marked sinus arrhythmia) can lead to errors in image quality (i.e., misregistration) due to reconstruction of images from incorrect phases of the cardiac cycle. Misregistration is seen as a projection in the reformatted sagittal and coronal views as an abrupt “jutt out” of the otherwise smooth edges of a normally sock shaped heart. Also in most cases, the extended region affects a set of images.

It would be desirable to provide a technique by which a reconstructed image of the heart and coronary vasculature could be generated while reducing the above described problems (i.e., misregistration) during prospective and retrospective ECG gating for cardiac imaging applications.

### BRIEF DESCRIPTION OF THE INVENTION

The above discussed and other drawbacks and deficiencies are overcome or alleviated by a system and method for calculating duration of a representative cardiac cycle using ECG waveform data. The method comprises generating the ECG waveform data using an electrocardiogram device and evaluating the ECG data to validate a signal from the electrocardiogram device. QRS complexes of ECG data are detected using a detection function and the underlying cardiac rhythm is analyzed based on the detected QRS complexes. An even number N of substantially normally shaped consecutive QRS complexes is selected and an RR interval between the consecutive QRS complexes is computed to yield N−1 intervals. The duration of the representative cardiac cycle is determined by averaging at least a plurality of the N−1 intervals, using either a median or mean method.

A system and method for improving image quality and dose reduction in the presence of arrhythmias during medical imaging with a scanning medical imaging system is also disclosed. The method comprises selecting a scanning window within a calculated representative R-R interval of the patient and scanning the patient's heart during the scanning window to obtain image data. An arrhythmia at one of prior and during the scanning window is detected and an image of the patient's heart representative of the scanning window of said R-R interval from chronologically discontinuous segments of the image data while rejecting any image data corresponding to any R-R interval of plurality of R-R intervals having said arrhythmia is assembled. The power applied to the scanning medical imaging system and translation of a table upon which the patient is disposed is optionally dependent upon detection of the arrhythmias.

The above discussed and other features and advantages of the present invention will be appreciated and understood by those skilled in the art from the following detailed description and drawings.

### BRIEF DESCRIPTION OF DRAWINGS

Referring to the exemplary drawings wherein like elements are numbered alike in the several Figures:

FIG. 1 is a perspective view of an ECG monitoring device communicated with a CT imaging system;

FIG. 2 is a block schematic diagram of the imaging system and ECG recording device illustrated in FIG. 1;

FIG. 3 is a representation of an ECG signal waveform;

FIG. 4 is a flow diagram describing a method for synchronizing an ECG waveform with a CT image, in accordance with an exemplary embodiment;

FIG. 5 is an example of an ECG waveform illustrating the synchronization timing between a CT exposure and a patient cardiac rhythm including a PVB before a scanning window, in accordance with an exemplary embodiment;

FIG. 6 is another example of an ECG waveform illustrating the synchronization timing between a CT exposure and a patient cardiac rhythm including a PVB during a scanning window, in accordance with another exemplary embodiment;

FIG. 7 is a legend for use with FIGS. 5 and 6;

FIG. 8 is an example of an ECG waveform illustrating the synchronization timing between a CT exposure and a patient cardiac rhythm including a PAB before a scanning window, in accordance with an exemplary embodiment;

FIG. 9 is another example of an ECG waveform illustrating the synchronization timing between a CT exposure and a patient cardiac rhythm including a PAB during a scanning window, in accordance with another exemplary embodiment; and

FIG. 10 is a legend for use with FIGS. 8 and 9.

### DETAILED DESCRIPTION OF THE INVENTION

As used herein, the term “tagging” means correlating, or associating, positional and/or cardiac phase data with the scan data. Such tagging is performed by storing the positional or cardiac phase data with the scan data itself (e.g., as a digital word) or by storing positional or cardiac phase data in a table that is correlated to the scan data. Additionally, the term “decomposing” means separating an ECG signal, or a portion of the ECG signal, into constituent parts, such as a P wave, a Q wave, an R wave, an S wave, or a T wave. As described herein, the ECG signal may be decomposed using a component delineation algorithm, such as the wavelet transform described in ‘Detection of ECG Characteristic Points Using Wavelet Transforms’, by C. Li, C. Zheng, and C. Tai, IEEE Transactions on Biomedical Engineering, Vol. 42, No. 1, January 1995.

Additionally, used herein, an element or step recited in the singular and preceded with the word “a” or “an” should be understood as not excluding plural said elements or steps, unless such exclusion is explicitly recited. Furthermore, references to “one embodiment” of the present invention are not intended to be interpreted as excluding the existence of additional embodiments that also incorporate the recited features. Also as used herein, the phrase “reconstructing an image” is not intended to exclude embodiments of the present invention in which data representing an image is generated but a viewable image is not. However, many embodiments generate (or are configured to generate) a viewable image.

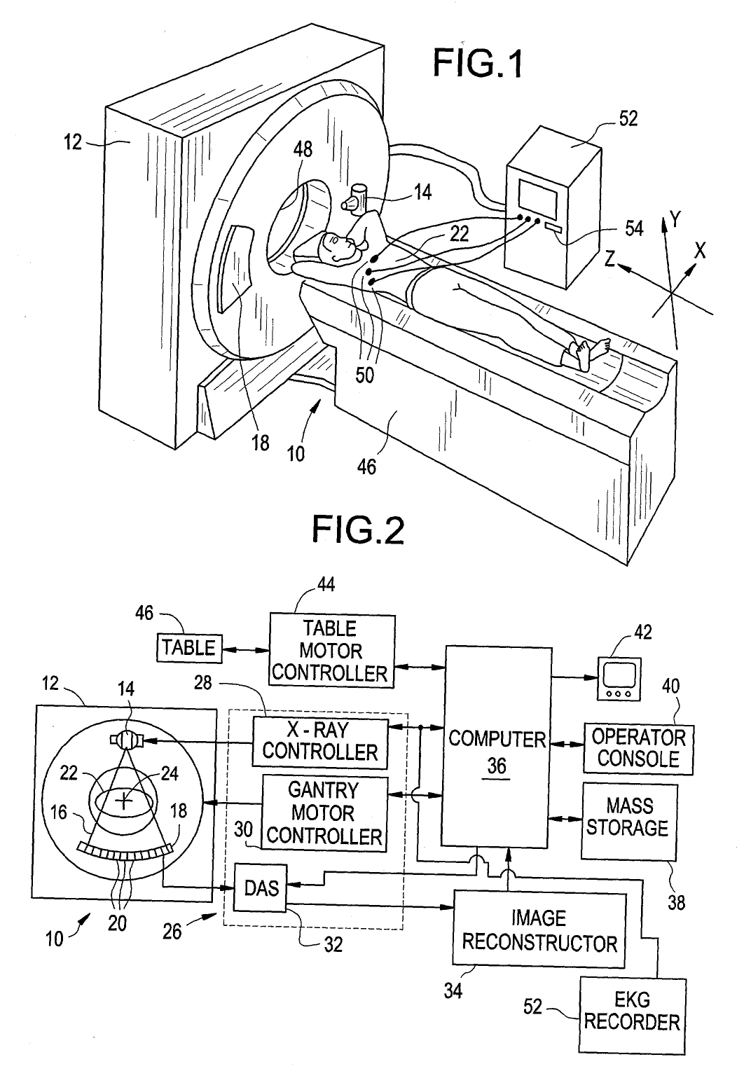

Referring to FIGS. 1 and 2, a computed tomography (CT) imaging system 10 is shown as including a gantry 12 representative of a “third generation” CT scanner. Gantry 12 has an x-ray source 14 that projects a beam of x-rays 16 toward a detector array 18 on the opposite side of gantry 12. Detector array 18 is formed by detector elements 20, which together sense the projected x-rays that pass through a medical patient 22. Each detector element 20 produces an electrical signal that represents the intensity of an impinging x-ray beam and hence the attenuation of the beam as it passes through patient 22. During a scan to acquire x-ray projection data, gantry 12 and the components mounted thereon rotate about a center of rotation 24.

Rotation of gantry 12 and the operation of x-ray source 14 are governed by a control mechanism 26 of CT system 10. Control mechanism 26 includes an x-ray controller 28 that provides power and timing signals to x-ray source 14 and a gantry motor controller 30 that controls the rotational speed and position of gantry 12. A data acquisition system (DAS) 32 in control mechanism 26 samples analog data from detector elements 20 and converts the data to digital signals for subsequent processing. An image reconstructor 34 receives sampled and digitized x-ray data from DAS 32 and performs high speed image reconstruction. The reconstructed image is applied as an input to a computer 36, which stores the image in a mass storage device 38, or outputs to a recording device (not shown), such as a film recorder. When an image is stored in storage device 38, the image may be stored as a data array, a linked list, or any other known data storage configurations. Computer 36 typically comprises a processor (not shown) and a memory device (not shown). The memory device may store a program, or algorithm, (not shown) comprising instructions for executing a process of the present invention. Alternatively, such a program may be executed, in whole or in part, by reconstructor 34, or by another computer system (not shown) included in, or coupled to, imaging system 10.

Computer 36 also receives commands and scanning parameters from an operator via console 40 that has a keyboard. An associated cathode ray tube display 42 allows the operator to observe the reconstructed image and other data from computer 36. In another embodiment, the reconstructed image may be transmitted as image data over a network (not shown) for disposition at another location. The operator supplied commands and parameters are used by computer 36 to provide control signals and information to DAS 32, x-ray controller 28 and gantry motor controller 30. In addition, computer 36 operates a table motor controller 44, which controls a motorized table 46 to position patient 22 in gantry 12. Particularly, table 46 moves portions of patient 22 through gantry opening 48.

The x-ray source and the detector array are rotated with a gantry within the imaging plane and around the object to be imaged so that the angle at which the x-ray beam intersects the object constantly changes. A group of x-ray attenuation measurements, i.e., projection data, from the detector array at one gantry angle and at a single axial position is referred to as a “view”. A “scan” of the object comprises a set of views made at different gantry angles, or view angles, during one revolution of the x-ray source and detector.

In an axial scan, the projection data is processed to construct an image that corresponds to a two-dimensional slice taken through the object. In a “helical” scan, the patient or object is moved while the data for the prescribed number of slices is acquired, thereby generating a single helix from a one fan beam helical scan. The helix mapped out by the fan beam yields projection data from which images in each prescribed slice may be reconstructed.

Cardiac reconstruction in multislice volume CT provides a three dimensional (3D) image of a beating heart at a given cardiac phase where the 3D volume is formed by a stack, or sequence, of parallel axial images. Helical scanning provides more axial coverage for a given patient breath-hold time. Therefore, the image reconstruction method and system described below are based on a protocol employing helical projections. However, the method and system are not limited to practice with helical scans, and other scan types, including, but not limited to axial and cine scans, can be employed. Additionally, system 10 is described herein by way of example only, and the image reconstruction method and system described below can be practiced in connection with many other types of imaging systems, for example, MR, ultrasound, PET, and nuclear scanners. Furthermore, the image reconstruction method, or algorithm, described herein is typically performed by image reconstructor 34. Such method, however, could be implemented in other components of the imaging system such as in computer 36.

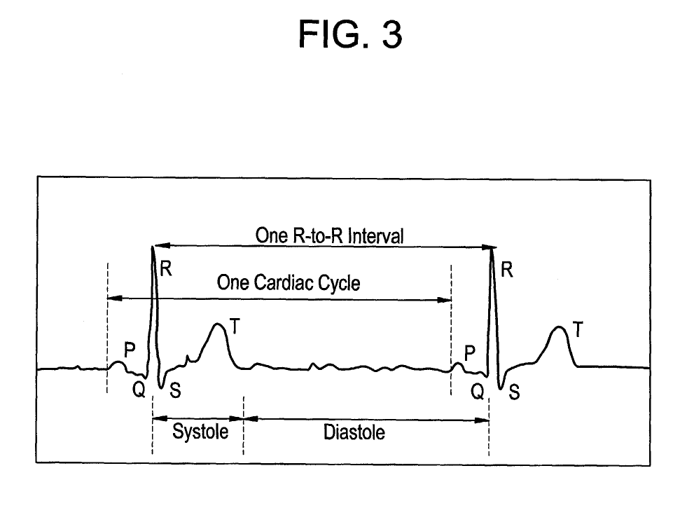

FIG. 3 illustrates one cardiac cycle for an ECG signal waveform, including a systolic phase—also known as systole, and a diastolic phase—also know as diastole, of the heart. The portions of the ECG signal labeled Q, R and S are referred to as a QRS complex, in which the R-feature, or R-wave, is typically the most prominent, highest amplitude, feature of the entire ECG signal. The cardiac cycle is typically defined as beginning with a P wave and continuing until the occurrence of a next P wave. An R-to-R interval—also known as ‘RR interval’—is defined as beginning with an R-wave and continuing until the occurrence of a next R-wave. The graphical representation of an ECG signal includes the QRS complex, a T wave, and a P wave. Analyzing the ECG signal with respect to the QRS complex, the T wave, and the P wave allows more accurate phase information to be correlated with projection data as the heart rate changes.

In one embodiment, imaging system 10 generates at least one image of an object in a defined condition, or state. For example, system 10 generates a series of images of a patient's beating heart in vivo. From the ECG signal QRS complex (often known as ‘R wave’) is detected using one of the several known methods. In one embodiment applying retrospective gating for example, a wavelet transform or a similar method is used to separate each RR interval into constituent parts, or wave components. More specifically, the wavelet transform decomposes each RR interval into P, Q, R, S, and T waves from which cardiac phase information is generated. The collected projection data are then tagged with the cardiac phase information. The tagged projection data are then used to reconstruct images, one per cardiac cycle or combined from multiple cardiac cycles using any of several known multi-sector algorithms to reconstruct an image, such as the filtered back projection technique described in, Principles of Computerized Tomographic Imaging, by A. C. Kak and M. Slaney, IEEE Press, New York N.Y., 1988.

The term cardiac state is used herein in relation to temporal points in the periodic cardiac motion that are defined with respect to the individual sub-waves, within the ECG signal. The term cardiac phase is used herein in relation to temporal points in the periodic cardiac motion that are defined only in relation to the R peaks. Both terms relate to temporal points in the periodic cardiac motion, and are only distinguished by the manner in which the temporal points are defined. Therefore, when the process of tagging projection data with cardiac phase information is discussed herein with respect to the present disclosure, the terms are interchangeable.

In the example embodiment, a cardiac CT scan is performed to acquire projection data from detector array 18 as table 46 moves a patient through gantry 12 at a fixed speed. A single projection data set produced by each detector element 20, for a given position of gantry 12, is generally referred to as a ‘view’. As projection data are acquired while table 46 moves in the z-direction, each view is correlated, or ‘tagged’, with z-location information. In one embodiment, computer 36 computes the z-location information and tags each view using information communicated from detector array 18, table motor controller 44 and gantry 12. For example, computer 36 utilizes the starting z-location of detector array 18, the period of gantry 12 and the table speed generated by motor controller 44 to compute a z-location for a corresponding view.

In addition to tagging each view with z-location information, system 10 tags each view with corresponding cardiac phase information. The corresponding cardiac phase information is determined by decomposing the ECG signal, collected simultaneously with the projection data, into a plurality of component waves and analyzing the component waves to determine cardiac state, or phase, information.

Therefore, in order to tag projection views with accurate cardiac phase information, system 10 determines cardiac phase information by analyzing the actual behavior, or motion, of the heart as projection data is acquired. This analysis allows projection views to be tagged with more accurate cardiac phase information. More specifically, for the image window following the current beat, desired phase is equal to a selected fraction of duration of the representative cardiac cycle determined by employing an exemplary method for definition of duration of the representative cardiac cycle described below. For retrospective gating, an algorithm is executed that utilizes wavelet transforms to analyze an ECG and determine the P, Q, R, S, and T waves within each cardiac cycle. In one embodiment, the algorithm is executed by reconstructor 34 (shown in FIG. 2) and stored in a storage device included in reconstructor 34. In an alternate embodiment, the algorithm is stored in mass storage device 38 (shown in FIG. 2) and executed by computer 36 (shown in FIG. 2).

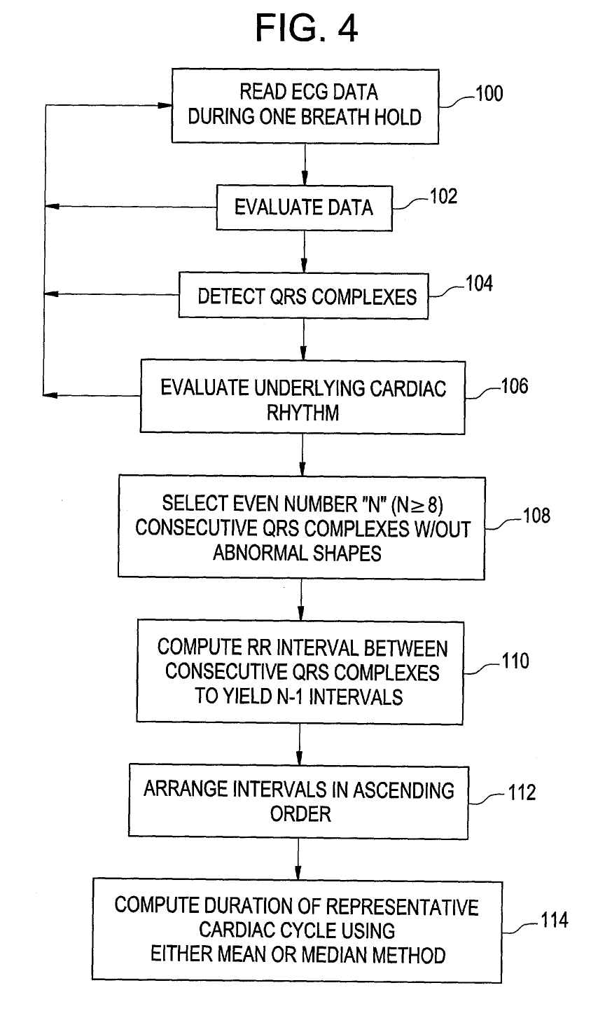

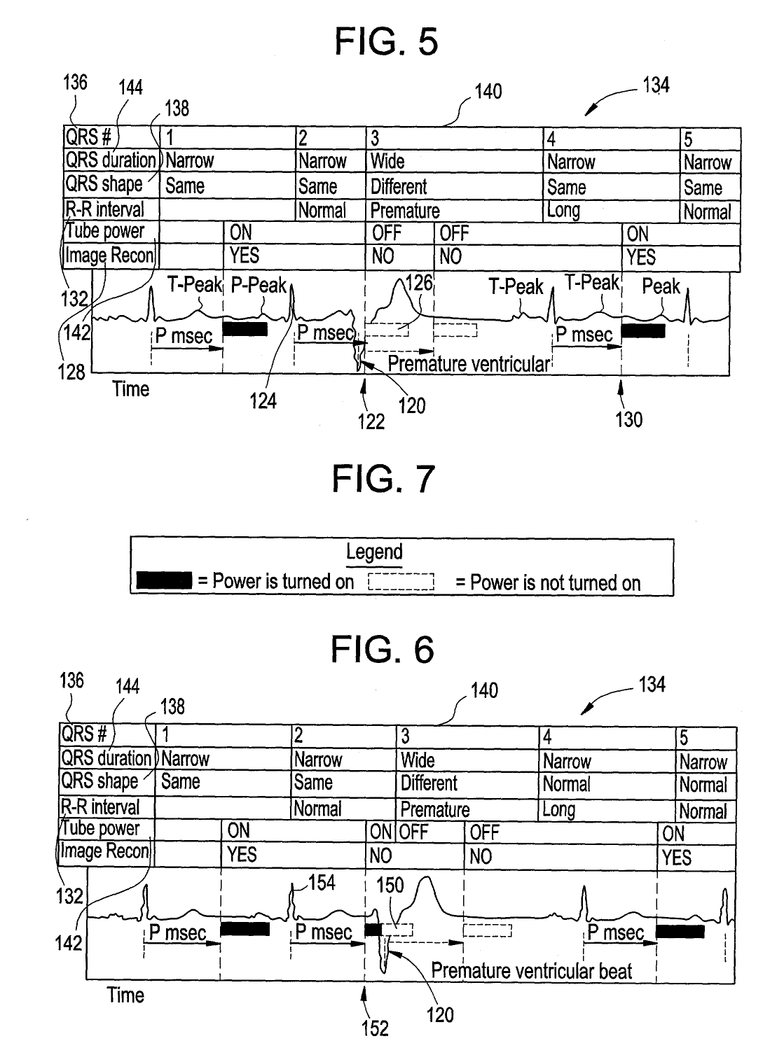

With respect to an exemplary method for duration of a representative cardiac cycle, FIGS. 5 and 6 represent an ECG signal and illustrates using a detection function to determine the QRS complexes using an absolute sum of filtered and first-differenced ECG data. More specifically with reference to the flowchart illustrated in FIG. 4, during a pre-scan or scout scan period, ECG data are acquired for about 20 to 30 seconds during one breath hold of the patient at block 100. ECG data are evaluated for noise, baseline stability and other possible artifacts at block 102. Based on the detected QRS duration and shape, premature ventricular beats (PVBs), premature atrial beats (PABs) and other abnormal beats (e.g., fusion beats, intermittent bundle block, and aberrantly conducted beats) are identified at block 104 and analysis of underlying cardiac rhythm is performed at block 106. If the rhythm is unsuitable for CT scanning as a result of a very high heart rate or bigeminy/trigeminy of PVBs, for example, a repeat ECG is acquired, processed and examined again. ECGs with occasional isolated PVBs are acceptable. If the cardiac rhythm is suitable, an even number “N” (N≧8) of consecutive QRS complexes without PVBs and other abnormal shaped waveforms are selected at block 108. The RR interval between consecutive QRS complexes is computed to yield N−1 intervals at block 110. After optionally first arranging the RR intervals in ascending order at block 112, duration of the representative cardiac cycle is further computed using one of the following two methods at block 114.

A first of the two methods is a mean method. In one embodiment, selection of a representative cardiac cycle duration includes discarding ‘M’ having the longest interval(s) and ‘M’ having the shortest interval(s) and compute the mean of the remaining intervals. The resultant mean value is a duration of the representative cardiac cycle to be used during scanning to determine the center of the scanning window.

A second of the two methods is a median method. In one embodiment, selection of a representative cardiac cycle duration includes selecting a middle or median value interval of the ascending orderly arranged RR interval distribution. The resultant median value is a duration of the representative cardiac cycle to be used during the scanning.

If abnormally shaped beats (PVBs and others) are present in the first N beats, another N consecutive beats are selected and all the above steps are repeated. If one or more abnormal beats are present in all possible sets of N consecutive beats, N+L consecutive beats (L≧2 and N+L must be an even number) are selected and any of the two above methods may be selected to calculate duration of a representative cardiac cycle.

Now referring to FIGS. 5-7, based on the representative cardiac cycle, desired phase(s) and width of imaging window, a beginning of the imaging window corresponding to the cardiac cycle delay (P msec) is determined. X-rays are turned on from the beginning to the end of the imaging window unless a premature beat is detected. More specifically, based on the representative cardiac cycle determined by one of the two averaging methods discussed above, a premature heart beat may be detected that occurs before or after the x-ray tube is powered during a particular imaging window. More specifically, having located the R-peaks in the selected frequency band, P msec delay following each R peak is specified. The size of the delay is based on the RR interval and the desired phase of the cardiac cycle for imaging. For example, the P msec delay may be 70% of the RR interval following a specific R peak. In retrospective gating, a window for search of end of T wave subsequent to each R peak is specified. For example, the T wave end search window may be 25% of the RR interval following each R peak. T wave end is identified by one of the several methods described in the pertinent scientific literature. The valley with the highest negative deflection between each P peak and R peak is identified as a Q wave, while the valley with the highest negative deflection between each T peak and R peak is identified as an S wave. Thus, by filtering the EKG signal into a frequency band, and identifying P, Q, R, S, and T peaks in the selected frequency band, cardiac states are accurately identified. Furthermore, accurate identification of cardiac states is maintained when the heart rate varies.

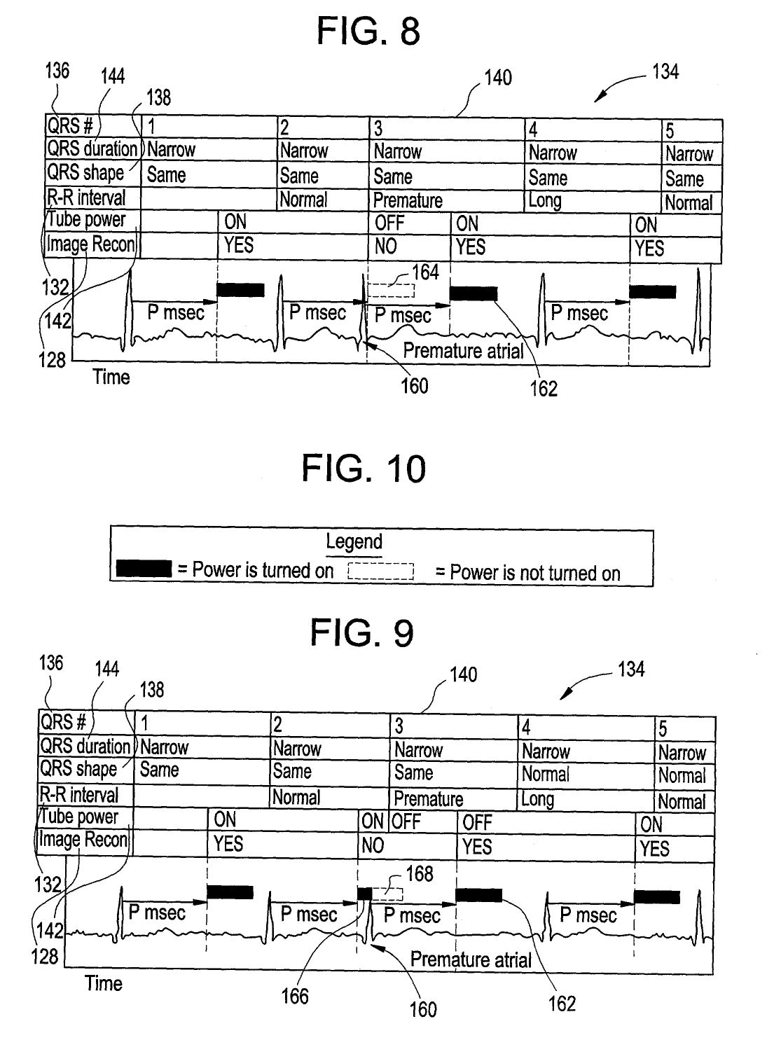

Referring to FIGS. 5 and 7, during prospective gating a premature ventricular beat (PVB) 120 is detected before a subsequent P msec 122 following a previous heartbeat 124. The X-ray tube is not powered as indicated by legend block 126, and scan data, collected if any, will be flagged “NO” as to “Image Recon” in row 128 such that data after this P msec 122 will not be considered for use in the reconstruction (see FIG. 8). Table movement is stopped until the next potentially useful cardiac cycle starts at P msec 130 and the table position is corrected, if needed. The X-ray tube is not powered during the next possible imaging window (heart beat following the PVB) because of the extent of ventricular filling is unpredictable following premature beats, particularly with PVBs. It will be recognized that unnecessary applied radiation dose is reduced by not powering the x-ray tube when any potential scan data will not be used in reconstruction.

FIG. 5 further illustrates R-R intervals 132 sectioned in table form generally at table 134. Each R-R interval 132 corresponds with a QRS #136 (i.e., 1−N) and a QRS shape 138 identified as “same” or “different.” A different QRS shape 138 corresponds with a PVB identified with QRS #3 in column 140. Column 140 also indicates that power to the x-ray tube is off as indicated in row 142 of table form 134 indicative of tube power being on or off during a QRS duration 144. QRS duration is identified as either being “narrow” or “wide” in table 134.

Referring now to FIGS. 6 and 7, another premature ventricular beat (PVB) 120 occurs after the x-ray tube is powered during a scanning window 150, i.e., during the image acquisition. The premature beat is detected after P msec 152 from a previous heartbeat 154, in which the x-ray tube has been already powered after P msec. Power to the x-ray tube is preferably turned off as soon as possible after the premature beat is detected to reduce the radiation dose applied to the patient unnecessarily. If this cannot be done, scanning with the x-ray tube on will run its course for the duration of scan window 130. In either case, scan data will be flagged to indicate to prevention its use in reconstruction. Table movement is stopped and table position corrected, if needed.

As in FIG. 5, FIG. 6 further illustrates R-R intervals 132 sectioned in table form generally at table 134. Each R-R interval 132 corresponds with QRS #136 (i.e., 1−N) and QRS shape 138 identified as “same” or “different.” A different QRS shape 138 corresponds with a PVB identified with QRS #3 in column 140. Column 140 also indicates that power to the x-ray tube is off as indicated in row 142 of table form 134 indicative of tube power being on or off during a QRS duration 144. QRS duration is identified as either being “narrow” or “wide” in table 134.

FIGS. 8 and 9 are examples with premature atrial beats (PABs) 160 very similar to that of FIGS. 5 and 6, respectively. The difference between a PAB (as in FIGS. 8 and 9) and a PVB (as in FIGS. 5 and 6) is that a PAB produces normal ventricular contraction and the following ventricular filling is similar to that of a beat occurring at normal R-to-R interval. Consequently, the x-ray tube will be powered in scanning windows 162 following a PAB and scan data are acquired and used in the image reconstruction as indicated in column 140 and row 128. FIGS. 8 and 9 also include corresponding tables 134 as in FIGS. 5 and 6.

More specifically, FIGS. 8 and 10 illustrate PAB 160 occurring just before QRS #3 and power is not turned on to the x-ray tube as indicated in scanning window 164. Consequently, there is no image data for image reconstruction and is correspondingly flagged as indicated in column 140, row 128. However, after P msec delay in QRS #3, power is turned on to x-ray tube in scanning window 162 and the scanned images are flagged for use in image reconstruction.

FIGS. 9 and 10 illustrate PAB 160 occurring just after a P msec delay in QRS #3 when power is turned on to the x-ray tube as indicated in scanning window 166. Consequently, scanned image data is flagged as “NO” in column 140, row 128 indicating rejection of such scanned images for use in image reconstruction. Furthermore, power is turned off to x-ray tube as indicated by a later portion 168 of scanning window 166. However, after P msec delay in QRS #3, power is turned on to x-ray tube in scanning window 162 and the scanned images are flagged for use in image reconstruction.

During scanning, based on analysis of the R-to-R interval, QRS duration and shape of a patient's ECG, appropriate flags are set as indicated in row 128 of table 134 indicative of whether the acquired scan data is suitable for use in the image reconstruction. If the flag indicates that data acquired during that cardiac cycle are suitable for reconstruction, they are used in the reconstruction of the required images. If the flag indicates otherwise, those data will not be used in reconstruction. If the data from an adjacent slab are overlapping, they may be used in reconstruction of the image corresponding to the missed slab or parts of it. If there is no overlap, the missed slab may be either left blank or reconstructed using interpolation techniques if acceptable. In any case, the image will be annotated to indicate that due to premature beats, image has been reconstructed with appropriate modifications to the reconstruction method.

In operation and prior to scanning with reference to the Figures, a patient is positioned on the scanner table 46, connected to the ECG monitor 52 and one or two trial ECG data acquisitions are made with breath-holding and other physiological maneuvers identical to those during the scan. ECG data are acquired during these trial acquisitions, ECG monitor is programmed to analyze the ECG waveform for determining measurements and morphology (e.g., shape) descriptors of the QRS such as: QRS duration and amplitude, polarity and duration of each of its components (e.g., Q wave, R wave, S wave), morphology of the QRS detection function (e.g., sum of absolute values of the first & second derivatives), amplitude and polarity of T wave and interval between the beginning of the QRS and peak of the T wave (i.e., Q-T peak interval). The ECG monitor if further programmed to calculate the representative R-to-R interval and detect presence of arrhythmias, including presence of premature beats. Results of this detailed analysis are sent to the scanner console 40 and utilized during the actual scan. Features of ECG during the scan are compared to features acquired during the trial period to determine nature of the incoming QRS complexes and T waves for proper control of power to X-ray tube and flags for inclusion/exclusion of data during image reconstruction, as described above with reference to examples of two types of premature beats monitored in an ECG: a) premature ventricular beat (PVB in FIGS. 5-7) and b) premature atrial beat (PAB in FIGS. 8-10). However, this method can be applied to prematurely with other arrhythmias such as atrial flutter with variable AV block, atrial fibrillation premature intermittent bundle branch blocks and aberrantly conducted beats.

Based on the duration of the representative cardiac cycle, desired phase(s) and width of an imaging window, the beginning of the imaging window with respect to a recent detected R wave is determined (i.e., a P msec delay). If the R-to-R interval is normal as in the case of the first two QRS complexes in FIGS. 5, 6, 8, and 9, the x-ray tube will be powered (see row 142 corresponding to ‘Tube power’ in table 134 just above a corresponding ECG waveform) at the selected phase and the acquired scan data are used in the image reconstruction (see row 128 corresponding to ‘Image Recon’ in table 134 just above a corresponding ECG waveform).

When the R-to-R interval is shorter than P msec as between the second QRS and the third QRS (a premature ventricular beat (PVB in FIG. 5 for example), the x-ray tube will not be powered. As a consequence, no data will be acquired and the image reconstruction will not take place. Table movement will be stopped until the next potentially useful cardiac cycle starts and its position is corrected, if needed. Since the PVB includes a wider QRS duration and a different QRS morphology compared to other normally conducted beats (See table 134 in FIG. 5), the x-ray tube will not be powered during the next possible imaging window because ventricular filling following a PVB will be different from that following a normal QRS.

With the embodiments described in this disclosure, there is a reduction of radiation dose for patients with arrhythmias and a significant improvement in image quality. As many patients undergoing cardiac tests (e.g., coronary artery imaging, ventricular function and cardiac perfusion) tend to be sick and arrhythmias are very common in such patients, it is advantageous to accurately scan nevertheless.

The above described method and system offers minimally invasive virtual cardiac catheterization and perfusion studies that reduces the risk of complications of an invasive catheterization and provide better quantitative resolution of perfusion with direct reference to cardiac anatomy. In contrast, nuclear imaging provides a relative perfusion image and does not provide any reference to cardiac anatomy. Furthermore, the exemplary method and system avoids unnecessary radiation dosage to the patient when any image produced as a result during or after a premature beat is not usable in reconstruction. The above method and system also provides more accurate estimation of the duration of the representative cardiac cycle to provide a better time reference for the image acquisition phase.

In accordance with an exemplary embodiment, processing of FIGS. 4, 6 and 7 may be implemented through processing device 40 operating in response to a computer program. In order to perform the prescribed functions and desired processing, as well as the computations therefore, the controller may include, but not be limited to, a processor(s), computer(s), memory, storage, register(s), timing, interrupt(s), communication interfaces, and input/output signal interfaces, as well as combinations comprising at least one of the foregoing. For example, the controller may include signal input signal filtering to enable accurate sampling and conversion or acquisitions of such signals from communications interfaces. It is also considered within the scope of the invention that the processing depicted in FIGS. 4, 6 and 7 may be implemented by a controller located remotely from processing device 40.

As described above, the present invention can be embodied in the form of computer-implemented processes and apparatuses for practicing those processes. The present invention can also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Existing systems having reprogrammable storage (e.g., flash memory) can be updated to implement the invention. The present invention can also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments configure the microprocessor to create specific logic circuits.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, the use of the terms first, second, etc. do not denote any order or importance, but rather the terms first, second, etc. are used to distinguish one element from another.

What is claimed is:

1. A method for calculating duration of a representative cardiac cycle using ECG waveform data, the method comprising:
generating the ECG waveform data using an electrocardiogram device;
evaluating said ECG data to validate a signal from said electrocardiogram device;
detecting QRS complexes of ECG data using a detection function;
analyzing underlying cardiac rhythm based on said detected QRS complexes;
selecting an even number N of substantially normally shaped consecutive QRS complexes;
computing an RR interval between said consecutive QRS complexes to yield N−1 intervals;
calculating duration of the representative cardiac cycle by averaging at least a plurality of said N−1 intervals.

2. The method of claim 1, wherein said selecting an even number N of substantially normally shaped consecutive QRS complexes includes N≧8.

3. The method of claim 1, wherein said validating said signal from said electrocardiogram device includes evaluating for at least one of noise, baseline stability, artifacts, including combinations of at least one of the foregoing.

4. The method of claim 1, wherein said analyzing underlying cardiac rhythm includes determination of a suitable heart rate.

5. The method of claim 1, wherein said calculating duration of the representative cardiac cycle by averaging includes averaging by one of a mean method and a median method.

6. The method of claim 5, wherein said mean method comprises:
discarding at least one of a longest and a shortest interval of said N−1 intervals; and
computing a mean of a remaining N−1 intervals indicative of the representative cardiac cycle so as to associate with a computed tomography imaging system scan.

7. The method of claim 6, wherein said discarding includes discarding said N−1 intervals≧1 second.

8. The method of claim 5, wherein said median method comprises:
arranging said N−1 intervals in ascending order; and
selecting a middle interval of said N−1 intervals in ascending order, said middle interval indicative of the representative cardiac cycle so as to associate with a computed tomography imaging system scan.

9. The method of claim 1, wherein said selecting an even number N of substantially normally shaped consecutive QRS complexes includes selecting N+L consecutive QRS complexes if abnormally shaped QRS complexes are present, where L≧2 and N+L is an even number.

10. A medium encoded with a machine-readable computer program code for associating ECG waveform data with medical imaging data using a data synchronization scheme, said medium including instructions for causing a controller to implement a method comprising:
generating the ECG waveform data using an electrocardiogram device;
evaluating said ECG data to validate a signal from said electrocardiogram device;
detecting QRS complexes of ECG data using a detection function;
analyzing underlying cardiac rhythm based on said detected QRS complexes;
selecting an even number N of substantially normally shaped consecutive QRS complexes;
computing an RR interval between said consecutive QRS complexes to yield N−1 intervals;
calculating duration of the representative cardiac cycle by averaging at least a plurality of said N−1 intervals.

11. The medium of claim 10, wherein said selecting an even number N of substantially normally shaped consecutive QRS complexes includes N≧8.

12. The medium of claim 11, wherein said validating said signal from said electrocardiogram device includes evaluating for at least one of noise, baseline stability, artifacts, including combinations of at least one of the foregoing.

13. The medium of claim 11, wherein said analyzing underlying cardiac rhythm includes determination of a suitable heart rate.

14. The medium of claim 11, wherein said calculating duration of the representative cardiac cycle by averaging includes averaging by one of a mean method and a median method.

15. The medium of claim 14, wherein said mean method comprises:
discarding at least one of a longest and a shortest interval of said N−1 intervals; and
computing a mean of a remaining N−1 intervals indicative of the representative cardiac cycle so as to associate with a computed tomography imaging system scan.

16. The medium of claim 15, wherein said discarding includes discarding said N−1 intervals≧1 second.

17. The medium of claim 14, wherein said median method comprises:
arranging said N−1 intervals in ascending order; and
selecting a middle interval of said N−1 intervals in ascending order, said middle interval indicative of the representative cardiac cycle so as to associate with a computed tomography imaging system scan.

18. The medium of claim 10, wherein said selecting an even number N of substantially normally shaped consecutive QRS complexes includes selecting N+L consecutive QRS complexes if abnormally shaped QRS complexes are present, where L≧2 and N+L is an even number.

19. A method for associating ECG waveform data with image data generated by an imaging system using a data synchronization scheme comprising:
obtaining the imaging system, an electrocardiogram device and an object to be examined;
associating said object with the imaging system and said electrocardiogram device; and
processing the image data and the ECG waveform data using the data synchronization scheme wherein the data synchronization scheme, generates the ECG waveform data using an electrocardiogram device;
evaluates said ECG data to validate a signal from said electrocardiogram device;
detects QRS complexes of ECG data using a detection function;
analyzes underlying cardiac rhythm based on said detected QRS complexes;
selects an even number N of substantially normally shaped consecutive QRS complexes;
computes an RR interval between said consecutive QRS complexes to yield N−1 intervals; and
calculates duration of the representative cardiac cycle by averaging at least a plurality of said N−1 intervals.

20. A system for associating ECG waveform data with image data using a data synchronization scheme comprising:
an imaging system;
an object disposed so as to be communicated with said imaging system, wherein said imaging system generates image data responsive to said object; and
a processing device having the data synchronization scheme, wherein the data synchronization scheme,
generates the ECG waveform data using an electrocardiogram device;
evaluates said ECG data to validate a signal from said electrocardiogram device;
detects QRS complexes of ECG data using a detection function;
analyzes underlying cardiac rhythm based on said detected QRS complexes;
selects an even number N of substantially normally shaped consecutive QRS complexes;
computes an RR interval between said consecutive QRS complexes to yield N−1 intervals; and
calculates duration of the representative cardiac cycle by averaging at least a plurality of said N−1 intervals.

21. The system of claim 20, wherein said object is a patient.

22. The system of claim 21, wherein said imaging system is a computed tomography imaging system.

23. A method to improve cardiac image quality in the presence of arrhythmias during medical imaging with a scanning medical imaging system, the method comprising:
calculating a representative R-R interval of a patient;
selecting a scanning window within said representative R-R interval;
scanning a patient's heart during said scanning window to obtain image data;
detecting an arrhythmia at one of prior and during said scanning window; and
assembling an image of the patient's heart representative of said scanning window of said R-R interval from chronologically discontinuous segments of the image data while rejecting any image data corresponding to any R-R interval of plurality of R-R intervals having said arrhythmia.

24. The method in accordance with claim 23 wherein scanning said patient's heart comprises axially scanning the patient's heart.

25. The method in accordance with claim 24 wherein said patient is supported on a moveable table, said axially scanning said patient's heart is repeated a plurality of times and said assembling said image of said patient's heart is repeated for each said axially scanning the patient's heart, and further comprising indexing said table between said axially scanning said patient's heart unless said arrhythmia is detected.

26. The method in accordance with claim 25 wherein power is turned on for said scanning during said scanning window after a delay from a start of said R-R interval.

27. The method in accordance with claim 26 wherein said power is turned off to prevent said scanning when said arrhythmia is detected during one of said delay and said scanning window.

28. The method in accordance with claim 27 wherein any image data obtained during said scanning window when said arrhythmia is detected is flagged for nonuse in image reconstruction.

29. The method in accordance with claim 28 wherein said table is one of not indexed and adjusted upon detection of said arrhythmia.

30. The method in accordance with claim 29, wherein in a contiguous succeeding R-R interval and after a second delay from said arrhythmia detected as a premature atrial beat (PAB), power to the imaging system is turned on during a second scanning window and an acquired image therefrom is flagged for use in said image reconstruction.

31. A scanning computed tomography (CT) imaging system for imaging a heart, said system configured to:
compute a representative R-R interval of a patient using an ECG signal;
scan said patient's heart a plurality of times during a selected scanning window of said R-R interval to obtain image data;
detect an arrhythmia at one of prior and during said scanning window;
reject any image data corresponding to any R-R interval having said arrhythmia; and
assemble an image of said patient's heart representative of said selected scanning window of the cardiac cycle from chronologically discontinuous segments of the image data.

32. The system in accordance with claim 31 further configured to axially scan the patient's heart.

33. The system in accordance with claim 32 further comprising a moveable table configured to support the patient, and wherein said system is further configured to perform a plurality of axial scans of the patient's heart, to assemble an image of the patient's heart for each of said plural axial scans, and to index said table between each of said axial scans unless said arrhythmia is detected.

34. The system in accordance with claim 33 further configured to turn power on for scanning during said scanning window after a delay from a start of said R-R interval.

35. The system in accordance with claim 34 further configured to turn power off to prevent scanning during said scanning window when said arrhythmia is detected when said arrhythmia is detected.

36. The system in accordance with claim 35 further configured to turn power on in a second scanning window in a succeeding contiguous R-R interval to said R-R interval and after a second delay from said arrhythmia detected as a premature atrial beat (PAB), wherein image data acquired from said second scanning window is flagged for use in said image reconstruction.
